(19) United States
(12) Patent Application Publication
(10) Pub. No.: US 2008/0194706 A1
(43) Pub. Date: Aug. 14, 2008

(54) Modified Open-Cell Foams and Method for Production Thereof

(75) Inventors: Ulrich Karl, Grunstadt (DE); Stefan Frenzel, Mannheim (DE); Volker Schwendemann, Neustadt (DE); Ulrich Steinbrenner, Neustadt (DE); Hartmut Leininger, Neustadt (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/720,289

(22) PCT Filed: Nov. 26, 2005
(86) PCT No.: PCT/EP05/12662

§ 371 Date: May 25, 2007

(30) Foreign Application Priority Data
Nov. 29, 2004 (DE) 10 2004 057 588.6
Jan. 24, 2005 (DE) 10 2005 003 316.4

(51) Int. Cl. C08J 9/30; A01P 1/00; A61K 8/72; C11D 3/37

(52) U.S. Cl. 514/772.3; 521/65; 510/475; 512/1

(57) ABSTRACT

Modified open-cell foams and method for the production thereof

Process for production of modified open-cell foams, which comprises bringing the following materials into contact
(a) open-cell foams with density in the range from 5 to 500 kg/m³ and average pore diameter in the range from 1 μm to 1 mm, and
(b) aqueous formulation of at least one compound having at least one hemiaminal or aminal group per molecule, or at least one copolymer comprising at least one copolymerized OH-containing or β-dicarbonyl-containing or epoxy-containing comonomer.

Modified open-cell foams and method for the production thereof

The present invention relates to a process for production of modified open-cell foams, which comprises bringing the following materials into contact

(a) open-cell foams with density in the range from 5 to 500 kg/m³ and average pore diameter in the range from 1 μm to 1 mm, and

(b) aqueous formulation of at least one compound having at least one hemiaminal or aminal group per molecule, or at least one copolymer comprising at least one copolymerized OH-containing or β-dicarbonyl-containing or epoxy-containing comonomer.

The present invention further relates to the use of inventive modified open-cell foams for production of cleaning materials, of filters, of humidifiers, of water distributors, of packaging elements, of sound-deadening elements such as vibration-inhibiting elements, or of buildings-insulation materials.

Foams, specifically those known as open-cell foams, are used in numerous applications. In particular open-cell foams composed of synthetic materials have proven versatile. By way of example, mention may be made of seat cushions, filter materials, air-conditioning systems, and automobile parts, and also cleaning materials.

Cleaning materials produced from foams are found, after relatively short service time, for example after about 10 minutes, to be damaged to the extent that no further cleaning action can be achieved. These cleaning materials moreover rapidly become unsightly. Producers of cleaning materials, such as wipers, therefore recommend disposal of cleaning materials after an appropriate service time which is generally very brief, e.g. 10 minutes, and this is a disadvantage for the consumer. Furthermore, delicate surfaces are often damaged by scratches, abrasion, or dulling.

U.S. Pat. No. 6,608,118 proposes compressing melamine foams with exposure to heat, for example compressing at 270° C. for 4 minutes. This gives foams with better mechanical properties and, by way of example, these are easy to divide.

EP 0 633 283 and DE 100 11 388 recommend reinforcing melalmine-resin foams by, for example, impregnating them with a silicone emulsion. However, silicone-emulsion-impregnated foams are not useful as cleaning materials because when used they leave residual streaks. DE 100 11 388 also recommends spraying melamine-resin foams with monomeric fluoroalkyl esters, in order to render them oil-repellent.

DE 102 20 896 proposes treating thermoset foam preforms with chemically inert binders which comprise an adhesive component. DE 102 09 601 discloses that addition of acid to thermoset foam preforms, such as melamine resin or phenolic resin, or else urea or bases can bring about hydrolysis, in particular acid-catalyzed hydrolysis of the resin.

However, the technical properties of the foams known from the prior art can be further improved in relation to cleaning action, stability, and water- or oil-absorption. Furthermore, the foams known from the prior art have proven to be insufficiently flexible in many instances.

It was therefore an object to provide foams which eliminate the disadvantages of the materials known from the prior art. Another object was to provide a process for production of novel foams. Another object was to provide uses for foams, and an object was to provide methods for the use of foams.

Accordingly, the process defined at the outset has been found. The process defined at the outset comprises processes for production of modified open-cell foams, which comprise bringing the following materials into contact

(a) open-cell foams with density in the range from 5 to 500 kg/m³ and average pore diameter in the range from 1 μm to 1 mm, and

(b) aqueous formulation of at least one compound having at least one hemiaminal or aminal group per molecule.

For the purposes of the present invention, aqueous formulation here can mean solutions, emulsions, or dispersions.

At least one compound in step (b) is preferably one compound which has not been used during production of unmodified foam (a).

In one embodiment of the present invention, the inventive open-cell modified foams are those based on synthetic organic foam, for example based on organic unmodified foams, such as foams based on polyurethane foams or aminoplastic foams, e.g. composed of urea-formaldehyde resins, or else foams based on phenol-formaldehyde resins, and in particular foams based on polyurethanes or aminoplastic-formaldehyde resins, in particular melamine-formaldehyde resins, and for the purposes of the present invention foams based on polyurethanes are also termed polyurethane foams, and foams based on melamine-formaldehyde resins are also termed melamine foams.

This means that the inventive foams are produced from open-cell foams which comprise synthetic organic materials, preferably polyurethane foams or aminoplastic foams, and in particular melamine foams.

For the purposes of the present invention, the unmodified open-cell foams (a) used to conduct the inventive process are very generally also termed unmodified foams (a). The unmodified open-cell foams (a) used to conduct the inventive process are described in more detail below.

The starting material used to conduct the inventive production process is open-cell foams (a), in particular foams in which at least 50% of the lamellae are open, preferably from 60 to 100%, and particularly preferably from 65 to 99.9%, determined to DIN ISO 4590.

The foams (a) used as starting material preferably comprise rigid foams, and for the purposes of the present invention these are foams whose compressive strength, determined to DIN 53577, is 1 kPa or more at 40% compression.

The density of foams (a) used as starting material is in the range from 5 to 500 kg/m³, preferably from 6 to 300 kg/m³, and particularly preferably in the range from 7 to 300 kg/m³.

The average pore diameter (number-average) of open-cell foams (a) used as starting material may be in the range from 1 μm to 1 mm, preferably from 50 to 500 μm, determined via evaluation of micrographs of sections.

In one embodiment of the present invention, the starting material used may comprise open-cell foams (a) having a maximum of 20, preferably a maximum of 15, and particularly preferably a maximum of 10, pores per m² whose diameter is in the range up to 20 mm. The other pores usually have a smaller diameter.

In one embodiment of the present invention, open-cell foams (a) used as starting material have a BET surface area in the range from 0.1 to 50 m²/g, preferably from 0.5 to 20 m²/g, determined to DIN 66131.

In one embodiment of the present invention, foams (a) used as starting material have a self-absorption level above 50%, measured to DIN 52215 at a frequency of 2000 Hz and at a layer thickness of 50 mm of the relevant foam (a).

In one specific embodiment of the present invention, open-cell foams (a) used as starting material have a sound-absorption level above 0.5, measured to DIN 52212 at a frequency of 2000 Hz and at a layer thickness of 40 mm of the relevant foam (a).

Open-cell foams (a) used as starting material may have any desired geometric shape, examples being sheets, spheres, cylinders, powders, cubes, flakes, blocks, saddles, bars, or round, rectangular, or square columns. The sizes of foams (a) used as starting material are non-critical, as long as they can be mechanically compressed by a machine. Preference is given to sheets, cylinders, cubes, blocks or rectangular columns, where these can be compressed mechanically in conventional apparatus.

One embodiment of the present invention starts from open-cell foams (a) composed of synthetic organic material, preferably from polyurethane foams or from melamine foams.

Polyurethane foams particularly suitable as starting material for carrying out the inventive process are known per se. By way of example, they are produced via reaction of

i) one or more polyisocyanates, i.e. compounds having two or more isocyanate groups,

ii) with one or more compounds having at least two groups reactive toward isocyanate, in the presence of

iii) one or more blowing agents,

iv) one or more starters,

v) and one or more catalysts, and

vi) cell openers.

Starters iv) and blowing agents iii) can be identical here.

Examples of suitable polyisocyanates i) are aliphatic, cycloaliphatic, araliphatic and preferably aromatic polyfunctional compounds known per se and having two or more isocyanate groups.

Specific examples are:

C₄-C₁₂-alkylene diisocyanates, preferably hexamethylene 1,6-diisocyanate; cycloaliphatic diisocyanates such as cyclohexane 1,3- and 1,4-diisocyanate and any mixtures of these isomers, 1-isocyanato-3,3,5-trimethyl-5-isocyanatomethyl-cyclohexane (isophorone diisocyanate, IPDI),

preferably aromatic diisocyanates and polyisocyanates such as tolylene 2,4- and 2,6-diisocyanate and corresponding isomer mixtures, diphenylmethane 4,4′-, 2,4′- and 2,2′-diisocyanate and corresponding isomer mixtures, mixtures of diphenylmethane 4,4′- and 2,4′-diisocyanates, polyphenyl polymethylene polyisocyanates, mixtures of diphenylmethane 4,4′-, 2,4′- and 2,2′-diisocyanates and polyphenyl polymethylene polyisocyanates (crude MDI), and mixtures of crude MDI with tolylene diisocyanates. Polyisocyanates can be used individually or in the form of mixtures.

Examples of ii) compounds having at least two groups reactive toward isocyanate are diols and polyols, in particular polyether polyols (polyalkylene glycols), these being prepared by methods known per se, for example by polymerization of one or more alkylene oxides, for example ethylene oxide, propylene oxide or butylene oxide, in the presence of alkali metal hydroxides as catalysts.

Very particularly preferred compounds ii) are ethylene glycol, propylene glycol, butylene glycol, 1,3-propanediol, 1,4-butanediol, 1,6-hexanediol, diethylene glycol, dipropylene glycol, triethylene glycol, tripropylene glycol, tetraethylene glycol, pentaethylene glycol, hexaethylene glycol.

Suitable blowing agents iii) are: water, inert gases, in particular carbon dioxide, and physical blowing agents. Physical blowing agents are compounds which are inert toward the starting components and are usually liquid at room temperature and vaporize under the conditions of the urethane reaction. The boiling point of these compounds is preferably below 110° C., in particular below 80° C. Among physical blowing agents are also inert gases which are introduced into the starting components i) and ii) or dissolved therein, for example carbon dioxide, nitrogen or noble gases.

Suitable compounds which are liquid at room temperature are usually selected from the group consisting of alkanes and/or cycloalkanes having at least 4 carbon atoms, dialkyl ethers, esters, ketones, acetals, fluoroalkanes having from 1 to 8 carbon atoms and tetraalkylsilanes having from 1 to 3 carbon atoms in the alkyl chain, in particular tetramethylsilane.

Examples which may be mentioned are: propane, n-butane, isobutane and cyclobutane, n-pentane, isopentane and cyclopentane, cyclohexane, dimethyl ether, methyl ethyl ether, methyl tert-butyl ether, methyl formate, acetone and fluorinated alkanes which can be degraded in the troposphere and therefore do not damage the ozone layer, e.g. trifluoromethane, difluoromethane, 1,1,1,3,3-pentafluorobutane, 1,1,1,3,3-pentafluoropropane, 1,1,1,2-tetrafluoroethane, 1,1,1-trifluoro-2,2,2-trichloroethane, 1,1,2-trifluoro-1,2,2-trichloroethane, difluoroethanes and heptafluoropropane. The physical blowing agents mentioned can be used either alone or in any combinations with one another.

The use of perfluoroalkanes for producing open cells is known from EP-A 0 351 614.

Examples of suitable starters iv) are: water, organic dicarboxylic acids, aliphatic and aromatic, if appropriate N-monoalkyl-, N,N- and N,N′-dialkyl-substituted diamines having from 1 to 4 carbon atoms in the alkyl radical, e.g. optionally N-monoalkyl- and N,N-dialkyl-substituted ethylenediamine, diethylenetriamine, triethylenetetramine, 1,3-propylenediamine, 1,3- or 1,4-butylenediamine, 1,2-, 1,3-, 1,4-, 1,5- and 1,6-hexamethylenediamine, aniline, phenylenediamines, 2,3-, 2,4-, 3,4- and 2,6-tolylenediamine and 4,4′-, 2,4′- and 2,2′-diaminodiphenylmethane.

Suitable catalysts v) are the catalysts known in polyurethane chemistry, for example tertiary amines such as triethylamine, dimethylcyclohexylamine, N-methylmorpholine, N,N′-dimethylpiperazine, 2-(dimethylaminoethoxy)ethanol, diazabicyclo[2.2.2]octane and the like and also, in particular, organic metal compounds such as titanic esters, iron compounds such as iron(III) acetylacetonate, tin compounds, e.g. tin diacetate, tin dioctoate, tin dilaurate or the dialkyl derivatives of dialkyltin salts of aliphatic carboxylic acids, e.g. dibutyltin diacetate and dibutyltin dilaurate.

Examples of cell openers vi) are polar polyether polyols (polyalkylene glycols) having high ethylene oxide content in the chain, preferably at least 50% by weight. These have a cell opening effect via demixing and effect on surface tension during foaming.

i) to vi) are used in the quantitative ratios customary in polyurethane chemistry.

Melamine foams particularly suitable as starting material for carrying out the inventive production process are known per se. By way of example, they are produced via foaming of

vii) a melamine-formaldehyde precondensate which may comprise other carbonyl compounds, such as aldehydes, co-condensed alongside formaldehyde,

viii) one or more blowing agents,

ix) one or more emulsifiers,

x) one or more hardeners.

Melamine-formaldehyde precondensates vii) may be unmodified precondensates, or else may be modified precondensates, and by way of example up to 20 mol % of the melamine may have been replaced by other thermoset-forming materials known per se, e.g. alkyl-substituted melamine, urea, urethane, carboxamides, dicyandiamide, guanidine, sulfuryl amide, sulfonamides, aliphatic amines, phenol, and phenol derivatives. Examples of other carbonyl compounds which may be present co-condensed alongside formaldehyde in modified melamine-formaldehyde precondensates are acetaldehyde, trimethylolacetaldehyde, acrolein, furfurol, glyoxal, phthalaldehyde and terephthalaldehyde.

Blowing agents viii) used may be the same as the compounds described in iii).

Emulsifiers ix) used may be conventional non-ionic, anionic, cationic, or betainic surfactants, in particular C₁₂-C₃₀-alkylsulfonates, preferably C₁₂-C₁₈-alkylsulfonates, and polyethoxylated C₁₀-C₂₀-alkyl alcohols, in particular having the formula R⁶—O(CH₂—CH₂—O)ₓ—H, where R⁶ is selected from C₁₀-C₂₀-alkyl and x may be, by way of example, a whole number in the range from 5 to 100.

Possible hardeners x) are, in particular, acidic compounds such as inorganic Brønsted acids, e.g. sulfuric acid or phosphoric acid, organic Brønsted acids such as acetic acid or formic acid, Lewis acids and also latent acids.

Examples of suitable melamine foams are described in EP-A 0 017 672.

Foams (a) used as starting material may, of course, also comprise additives customary in foam chemistry, for example antioxidants, flame retardants, fillers, colorants such as pigments or dyes, and biocides, such as

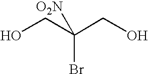

The present invention also starts from at least one compound having at least one hemiaminal or aminal group per molecule, or at least one copolymer comprising at least one copolymerized OH-containing or β-dicarbonyl-containing or epoxy-containing comonomer, or comprising copolymerized n-butyl acrylate.

The abbreviated terms “compound (b)” or “(b)” are also used below for compounds used having at least one hemiaminal or aminal group per molecule and copolymers comprising at least one copolymerized OH-containing or β-dicarbonyl-containing or epoxy-containing comonomer or comprising copolymerized n-butyl acrylate. By way of example, compound (b) is obtainable via condensation of at least one nitrogen-containing compound (B1) and of at least one carbonyl compound (B2), and, if appropriate, of other compounds (B3), and, if appropriate, further reactions after the condensation process.

Examples of nitrogen-containing compounds (B1) are urea, N,N′-dimethylurea, triazones, tetrahydropyrimidinones, imidazolinones, tetrahydro-4H-1,3,5-oxadiazin-4-ones, alkylcarbamates, methoxyethylcarbamates, and methylol(meth)acrylamide.

Examples of carbonyl compounds (B2) are
ketones, in particular di(C₁-C₁₀-alkyl) ketones,
preferably mono-, di- and polyaldehydes, in particular C₁-C₁₀-alkyl monoaldehydes,
such as acetaldehyde or propionaldehyde, and very particularly preferably formaldehyde, and also dialdehydes, such as glyoxal or phthalaldehyde, e.g. 1,2-phthalaldehyde, butanedial, glutaraldehyde and hexane-1,6-dial.

Examples of particularly preferred other compounds (B3) are mono- or polyhydric alcohols, such as C₁-C₁₀ alkanols, in particular methanol, ethanol, n-propanol and n-butanol, and also ethylene glycol, propylene glycol, butylene glycol, 1,4-butanediol, 1,6-hexanediol, 1,12-dodecanediol, glycerol, diethylene glycol, dipropylene glycol, polyethylene glycols having an average of up to 200, preferably from 3 up to 20, ethylene oxide units per molecule (number average), polypropylene glycols having an average of up to 200, preferably from 3 up to 20, propylene oxide units per molecule (number average), polytetrahydrofuran having an average of up to 200, preferably from 3 up to 20, 1,4-butanediol units per molecule (number average), and also mono-C₁-C₁₀-alkyl-capped mono-, di- or polyethylene or -propylene glycols having an average of up to 200, preferably from 3 up to 20, alkylene oxide units per molecule (number average).

Examples of further reactions after the condensation process are esterification processes, etherification processes, and free-radical (co)polymerization processes.

In one embodiment of the present invention, compound (b) may be prepared from at least one nitrogen-containing compound (B1), from at least two carbonyl compounds (B2), and, by way of example, from up to 3 different other compounds (B3).

Particularly preferred examples of compounds (b) are those of the general formula I a to I b

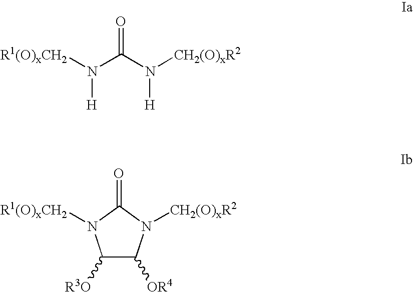

the variables being defined as follows:
R¹ and R² are different or preferably identical and are selected from hydrogen, C₁-C₁₂-alkyl, branched or unbranched, selected from methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, isoheptyl, n-octyl, n-nonyl, n-decyl, and n-dodecyl; preferably C₁-C₆-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, particularly preferably C₁-C₄-alkyl, such as methyl, ethyl, n-propyl, isopropyl, n-butyl, isobutyl, sec-butyl and tert-butyl,
   (—CH₂—CH₂—O)ₘ—R⁵, (—CHCH₃—CH₂—O)ₘ—R⁵, (—CH₂—CHCH₃—O)ₘ—R⁵,
   (—CH₂—CH₂—CH₂—O)ₘ—R⁵, (—CH₂—CH₂—CH₂—CH₂—O)ₘ—R⁵,
x are identical or different and are a whole number selected from zero and one, at least one x in formula I a being selected to be equal to one; both x in formula I b may be selected to be equal to zero,
m is a whole number in the range from 1 to 20,
R³ and R⁴ are different or preferably identical and are selected from hydrogen, C₁-C₁₂-alkyl, branched or unbranched, selected from methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, isoheptyl, n-octyl, n-nonyl, n-decyl and n-dodecyl; preferably C₁-C₆-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, particularly preferably C₁-C₄-alkyl, such as methyl, ethyl, n-propyl, isopropyl, n-butyl, isobutyl, sec-butyl, and tert-butyl, or together are C₂-C₄-alkylene, such as —CH₂—CH₂—, —(CH₂)₃—, or —(CH₂)₄—,
R⁵ are identical or different and are selected from C₁-C₄-alkyl, such as methyl, ethyl, n-propyl, isopropyl, n-butyl, isobutyl, sec-butyl and tert-butyl, and in particular hydrogen.

Compounds (b) in particular of the general formula I a and I b are known per se. Compounds (b) particularly of the general formula I a and I b are generally not in the pure form defined by a formula; intermolecular rearrangements of the radicals R¹ to R⁴ are usually found to occur, examples being transaminalization reactions, and condensation reactions and cleavage reactions are also found to occur to a certain extent. The formula I a or I b given above is to be interpreted as defining the stoichiometric ratios of the substituents and also comprising intermolecular rearrangement products and condensates.

Another group of compounds (b) preferably used is that of homo- and in particular copolymers of compounds of the general formula II

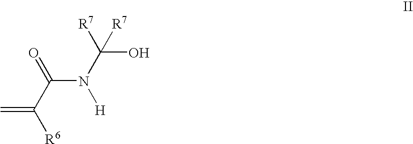

where the variables are defined as follows:
R⁶ is selected from hydrogen and C₁-C₁₂-alkyl, preferably linear C₁-C₁₂-alkyl, selected from methyl, ethyl, n-propyl, n-butyl, n-pentyl, n-hexyl, n-heptyl, n-octyl, n-nonyl, n-decyl and n-dodecyl; preferably linear C₁-C₆-alkyl, such as methyl, ethyl, n-propyl, n-butyl, n-pentyl, isopentyl, n-hexyl, particularly preferably C₁-C₄-alkyl, such as methyl, ethyl, n-propyl, and n-butyl, very particular preference being given here to hydrogen and methyl,
R⁷ are different or preferably identical, and are selected from C₁-C₁₂-alkyl, preferably linear C₁-C₁₂-alkyl, selected from methyl, ethyl, n-propyl, n-butyl, n-pentyl, n-hexyl, n-heptyl, n-octyl, n-nonyl, n-decyl and n-dodecyl; preferably linear C₁-C₆-alkyl, such as methyl, ethyl, n-propyl, n-butyl, n-pentyl, isopentyl, n-hexyl, particularly preferably C₁-C₄-alkyl, such as methyl, ethyl, n-propyl, and n-butyl, and particularly preferably hydrogen.

Both variables R⁷ in formula II are very particularly preferably hydrogen, R⁶ being very particularly preferably selected from methyl and hydrogen.

By way of example, the molar masses M_{w} of homo- and copolymers preferably used of compounds of the general formula II may be from 10,000 to 250,000 g/mol, preferably from 20,000 to 240,000 g/mol.

If it is desired to use copolymers of one or more compounds of the general formula II, those which may be used are in particular copolymers of one or more compounds of the general formula II with one, or preferably at least two, comonomers, selected from one or more C₁-C₁₀-alkyl (meth)acrylates, in particular methyl acrylate, ethyl acrylate, n-butyl (meth)acrylate, 2-ethylhexyl (meth)acrylate,
(meth) acrylic acid,
vinylaromatic compounds, such as styrene,
(meth)acrylonitrile, and
(meth)acrylamide.

If it is desired to use a copolymer comprising at least one copolymerized OH-containing or β-dicarbonyl-containing or epoxy-containing comonomer, or comprising copolymerized n-butyl acrylate, it is preferable to use copolymers which comprise at least one copolymerized comonomer of the general formula III

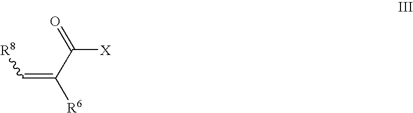

where the variables are defined as follows:
R⁸ is selected from C₁-C₁₂-alkyl, preferably linear C₁-C₁₂-alkyl, selected from methyl, ethyl, n-propyl, n-butyl, n-pentyl, n-hexyl, n-heptyl, n-octyl, n-nonyl, n-decyl and n-dodecyl; preferably linear C₁-C₆-alkyl, such as methyl, ethyl, n-propyl, n-butyl, n-pentyl, isopentyl, n-hexyl, particularly preferably C₁-C₄-alkyl, such as methyl, ethyl, n-propyl and n-butyl,

and very particularly preferably hydrogen,
X is selected from OH, glycidyl, 2-hydroxyethyl, 3-hydroxypropyl,

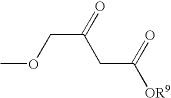

where
R⁹ is selected from C₁-C₁₂-alkyl, branched or unbranched, selected from methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, isoheptyl, n-octyl, n-nonyl, n-decyl and n-dodecyl, preferably C₁-C₆-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, particularly preferably C₁-C₄-alkyl, such as methyl, ethyl, n-propyl, isopropyl, n-butyl, isobutyl, sec-butyl, and tert-butyl, very particularly preferably methyl.

If it is desired to use, as (b), copolymers which comprise a copolymerized compound of the general formula III, where X═OH, preference is then given to copolymers which do not comprise ethylene as copolymerized comonomer.

In one embodiment of the present invention, the copolymers selected as (b) comprise those which comprise the following copolymerized compounds:
from 0 to 15% by weight, preferably from 0.5 to 10% by weight, of at least one comonomer of the general formula II or III,
from 0 to 80% by weight of n-butyl acrylate,
from 0 to 80% by weight of at least one other C₁-C₁₀-alkyl(meth)acrylate,
from 0 to 20% by weight, preferably from 0.1 to 15% by weight, of one or more other comonomers, such as (meth)acrylic acid, vinylaromatic compounds, such as styrene, (meth)acrylonitrile, and (meth)acrylamide.

If it is desired to use copolymeric compounds of the general formula II or III, it is preferable to use random copolymers, which can be prepared by methods known per se, for example via emulsion polymerization.

In one embodiment of the present invention, aqueous formulations used in step (b) comprise an amount in the range from 1 to 60% by weight, preferably from 10 to 40% by weight, of compound (b).

Various techniques could be used to bring unmodified foams (a) into contact with compound (b).

By way of example, the contact may be brought about via immersion of unmodified foam (a) in aqueous formulation of compound (b), via impregnation of unmodified foam (a) with aqueous formulation of compound (b), via saturation of unmodified foam (a) with aqueous formulation of compound (b), via partial or preferably entire spraying of unmodified foam (a) with aqueous formulation of compound (b), or via calendering to apply aqueous formulation of compound (b) to unmodified foam (a).

In another embodiment of the present invention, the method of working the inventive process is that aqueous formulation of compound (b) is applied to unmodified foam (a) by doctoring. After saturation or application by doctoring or application by calendering or spraying, a squeezing process between at least two rolls, for example rotating rolls, may be used to remove liquid and thus attain uniform distribution of the formulation and set the desired concentration.

In one embodiment of the present invention, after the contacting process, unmodified foam (a) and aqueous formulation of compound (b) may be permitted to interact, for example over a period in the range from 0.1 second to 24 hours, preferably from 0.5 second to 10 hours, and particularly preferably from 1 second to 6 hours.

In one embodiment of the inventive production process, unmodified foam (a) and aqueous formulation of compound (b) are brought into contact at temperatures in the range from 0° C. to 250° C., preferably from 5° C. to 190° C., and particularly preferably from 10 to 180° C.

In one embodiment of the inventive production process, unmodified foam (a) and aqueous formulation of compound (b) are initially brought into contact at temperatures in the range from 0° C. to 50° C. and the temperature is then changed, for example by heating to temperatures in the range from 60° C. to 250° C., preferably from 65° C. to 180° C.

In another embodiment of the inventive production process, unmodified foam (a) and aqueous formulation of compound (b) are initially brought into contact at temperatures in the range from 0° C. to 120° C. and the temperature is then changed, for example by heating to temperatures in the range from 30° C. to 250° C., preferably from 125° C. to 200° C.

In one preferred embodiment of the present invention, the amounts selected of the starting materials: unmodified foam (a) and aqueous formulation of compound (b) are such that inventive product has markedly higher density than the corresponding unmodified foam (a).

In one embodiment of the present invention, the inventive process is carried out at atmospheric pressure while unmodified foam (a) is brought into contact with aqueous formulation of compound (b). In another embodiment of the present invention, the inventive process is carried out by operating at an elevated pressure, for example at pressures in the range from 1.1 bar to 10 bar. In another embodiment of the present invention, the inventive process is carried out by operating at a reduced pressure, for example at pressures in the range from 0.1 mbar to 900 mbar, preferably up to 100 mbar.

In one embodiment of the present invention, unmodified foam (a) is brought into contact with aqueous formulation of compound (b) in such a way as to distribute compound (b) with maximum uniformity in all dimensions over unmodified foam (a). Suitable methods are methods with high application effectiveness. Examples which may be mentioned are: complete saturation, immersion, flow coating, drum application, spray application, e.g. compressed-air spraying, airless spraying, and also atomization at high rotation rates, coating, application by doctoring, application by calendering, spreading, roller-application, application by wiping, rolling methods, spinning methods, and centrifuging methods.

In another embodiment of the present invention, unmodified foam (a) is brought into contact with aqueous formulation of compound (b) in such a way as to bring about non-uniform distribution of aqueous formulation of compound (b) on unmodified foam (a). By way of example, in one embodiment of the present invention it is possible to spray aqueous formulation of compound (b) non-uniformly onto unmodified foam (a) and then allow the materials to interact. In another embodiment of the present invention, unmodified foam (a) may be partially saturated with aqueous formulation of compound (b). In another embodiment of the present invention, a portion of unmodified foam (a) may be brought into contact once, and another portion of unmodified foam (a) at least twice, with aqueous formulation of compound (b). In another embodiment, unmodified foam (a) is saturated completely with aqueous formulation of compound (b), and the uppermost layer is rinsed clean again with, for example, water. The materials are then allowed to interact. The result is coating within the core of unmodified foam (a); the outer surface remains uncoated.

If unmodified foam (a) is brought into contact with aqueous formulation of compound (b) in a way that has brought about non-uniform distribution of aqueous formulation of compound (b) on unmodified foam (a), an example of a result achieved is that, by allowing the materials to interact over a period of 2 minutes or more, it is not only the outermost layer of unmodified foam (a) that is brought into contact with aqueous formulation of compound (b).

If unmodified foam (a) is brought into contact with aqueous formulation of compound (b) in such a way that non-uniform distribution of aqueous formulation of compound (b) has been brought about on unmodified foam (a), a possible result is that inventively modified foam has non-uniform mechanical properties over its cross section. By way of example, it is possible according to the invention that it is softer at the sites where it has been brought into contact with relatively large amounts of aqueous formulation of compound (b) than at the sites where it has been brought into contact with less aqueous formulation of compound (b).

In one embodiment of the present invention, in some instances where non-uniform distribution of the aqueous formulation of compound (b) is not desirable per se, it can be rendered more uniform via calendering on perforated rolls or on perforated metal sheets. A preferred method of reducing the extent of non-uniform distribution of aqueous formulation of compound (b), uses at least two perforated rolls, by applying vacuum suction on at least one perforated roll or at least one perforated metal sheet.

In one specific embodiment of the present invention, a defined liquor absorption level is set after the materials have been brought into contact, via squeezing between two counter-rotating rolls, to remove liquid, an example of the defined liquor absorption level being from 20 to 800% by weight, based on the weight of the unmodified foam (a). The concentration of compound (b) in the formulation is from 1 to 99% by weight.

In one embodiment of the present invention, after the materials have been brought into contact, rinsing may be carried out, for example using one or more solvents, and preferably using water.

In one embodiment of the present invention, after the materials have been brought into contact and, if appropriate, after rinsing, drying may be carried out, for example mechanically via, by way of example, wringing or calendering, in particular by using two rollers to remove liquid by squeezing, or thermally, for example in microwave ovens, hot-air-blower systems, or drying cabinets, in particular vacuum drying cabinets, the temperatures at which drying cabinets may be operated being, by way of example, from 30 to 150° C. In the context of vacuum drying cabinets vacuum can be interpreted as a pressure in the range from 0.1 to 850 mbar, for example.

The time taken for drying steps carried out if desired is defined as not included in the interaction time for the purposes of the present invention.

In one embodiment of the present invention, thermal drying may be brought about via heating to temperatures in the range from 20° C. to 150° C., for example over a period of from 10 seconds to 20 hours.

According to the invention, unmodified foam (a) may be brought into contact not only with aqueous formulation of compound (b) but also with at least one catalyst (c). Examples of suitable compounds are metal salts, ammonium salts, and inorganic or organic acids. Examples of suitable metal salts are metal halides, metal sulfates, metal nitrates, metal tetrafluoroborates, metal phosphates, or a mixture of these. Examples are magnesium chloride, magnesium sulfate, zinc chloride, lithium chloride, lithium bromide, boron trifluoride, aluminum chloride, aluminum sulfate, alums, such as KAI(SO₄)₂.12 H₂O, zinc nitrate, sodium tetrafluoroborate, and mixtures of the metal salts described above.

Ammonium salts suitable as catalyst (c) are ammonium salts from the group of ammonium chloride, ammonium nitrate, ammonium sulfate, ammonium oxalate, diammonium phosphate, and mixtures of the ammonium salts described above.

Inorganic and organic acids suitable as catalyst (c) are maleic acid, formic acid, citric acid, tartaric acid, oxalic acid, p-toluenesulfonic acid, hydrochloric acid, sulfuric acid, boric acid, and mixtures of these.

It is also possible, of course, to use mixtures of, by way of example, at least one metal salt and at least one ammonium salt, or at least one metal or ammonium salt and at least one organic or inorganic acid, as catalyst (c).

Very particularly preferred catalysts (c) are Brønsted acid catalysts, such as ZnCl₂, Zn(NO₃)₂, each of these also in the form of their hydrates, NH₄Cl, MgSO₄, Al₂(SO₄)₃, each of these also in the form of their hydrates, and very particularly preferably MgCl₂, in particular in the form of its hexahydrate.

Based on compound (b), it is preferable to use one third to one twentieth of the weight of catalyst (c), in each case determined without any water of hydration present.

Preference is given to magnesium chloride, zinc chloride, magnesium sulfate, aluminum sulfate. Magnesium chloride is particularly preferred.

In one embodiment of the present invention, unmodified foam (a) is brought into contact with aqueous solution of compound (b) and, if appropriate, catalyst (c) at pH in the range from 3.0 to 7.5, and the desired pH here can be set, if appropriate, via addition of acid, alkali, or buffer. It is preferable to use a buffer.

In one embodiment of the present invention, at least one unmodified foam (a) may be brought into contact not only with aqueous formulation of compound (b) and, if appropriate, catalyst (c), but also with at least one additive (d), selected from biocides, such as silver particles or monomeric or polymeric organic biocides, such as phenoxyethanol, phenoxypropanol, glyoxal, thiadiazines, 2,4-dichlorobenzyl alcohols, and preferably isothiazolone derivatives, such as MIT (2-methyl-3(2H)-isothiazolone), CMIT (5-chloro-2-methyl-3(2H)-isothiazolone), CIT (5-chloro-3(2H)-isothiazolone), BIT (1,2-benzoisothiazol-3(2H)-one), and also copolymers of N,N-di-C₁-C₁₀-alkyl-ω-amino-C₂-C₄-alkyl (meth)acrylate, in particular copolymers of ethylene with N,N-dimethyl-2-aminoethyl (meth)acrylate,
one or more surfactants, which may be anionic, cationic, or nonionic,
activated charcoal,
colorants, such as dyes or pigments,
fragrances, e.g. perfume,
hydrophobicizers or oleophobicizers, such as fluorocarbon resins or fluorocarbon waxes,
odor scavengers, such as cyclodextrins, and
   microcapsules, charged with at least one active ingredient, such as treatment oil, with one or more biocides, perfume, or odor scavenger, and for the purposes of the present invention the microcapsules may be, by way of example, spherical hollow particles with an average external diameter in the range from 1 to 100 μm, which may be composed, by way of example, of melamine-formaldehyde resin or of polymethyl methacrylate.

An example of a procedure for this brings at least one unmodified foam (a) into contact with aqueous formulation of compound (b) and with at least one additive (d) in various operations or preferably simultaneously.

In one embodiment of the present invention, one or more additives (d) may be added to aqueous formulation of compound (b), for example in proportions of from 0 to a total of 50% by weight, based on (b), preferably from 0.001 to 30% by weight, particularly preferably from 0.01 to 25% by weight, very particularly preferably from 0.1 to 20% by weight.

In another method of carrying out the inventive process, after aqueous formulation of compound (b) and, if appropriate, catalyst (c), and, if appropriate, at least one additive (d) have been allowed to act on unmodified foam (a), mechanical compression may be exerted one or more times. The mechanical compression may be exerted batchwise or preferably continuously, for example batchwise via presses or plates, or continuously via rolls or calenders, for example. If calendering is desired, one or more calender passes may be carried out, for example from one to twenty calender passes, preference being given to from five to ten calender passes.

In one embodiment of the present invention, mechanical compression is carried out to a degree of compaction in the range from 1:1.2 to 1:20, preferably from 1:2.5 to 1:10.

In one embodiment of the present invention, calendering is carried out prior to the drying process.

In one embodiment of the present invention, the procedure is that after aqueous formulation of compound (b) and, if appropriate, catalyst (c), and if appropriate, at least one additive (d) have been brought into contact and allowed to interact, the product is first dried, then moistened with water, and then mechanically compressed, for example calendered.

In another embodiment of the present invention, the procedure is that after aqueous formulation of compound (b) and, if appropriate, catalyst (c), and, if appropriate, at least one additive (d) have been brought into contact and allowed to interact, the product is first dried, the moistening process is omitted, and then the product is mechanically compressed, for example calendered.

In one embodiment of the present invention, after aqueous formulation of compound (b), and, if appropriate, catalyst (c), and, if appropriate, at least one additive (d) have been brought into contact and allowed to interact, the mechanical compression process produces soft and flexible foams from the unmodified foams (a) which are per se rigid.

In one embodiment of the present invention, after aqueous formulation of compound (b) and, if appropriate, catalyst (c), and, if appropriate, at least one additive (d) have been brought into contact and allowed to interact, heat-setting may be carried out on unmodified foam (a), and specifically prior to or after the mechanical compression process, or else between two mechanical compression steps. By way of example, heat-setting may be carried out at temperatures of from 120° C. to 250° C. over a period of from 5 seconds to 120 minutes. Examples of suitable apparatus are microwave ovens, plate press systems, drying cabinets heated by hot-air-blower systems, or by electricity or by gas flames, heated roll mills, or continuously-operated drying equipment.

Drying, as described above, may be carried out prior to the heat-setting process.

In one embodiment of the present invention, after aqueous formulation of compound (b) and, if appropriate, catalyst (c), and, if appropriate, at least one additive (d) have been brought into contact and allowed to interact, heat-setting may be carried out on unmodified foam (a), and specifically after or preferably prior to the mechanical compression process, or else between two mechanical compression steps. By way of example, heat-setting may be carried out at temperatures of from 150° C. to 200° C. over a period of from 30 seconds to 120 minutes. Examples of suitable apparatus are drying cabinets.

In one specific embodiment, the mechanical compression process and the heat-setting process are combined, for example after the materials have been allowed to interact and, if appropriate, after the drying process, by passing the foam one or more times over hot rolls or calenders, or compressing it one or more times between hot plates. It is also possible, of course, to calender two or more times and during this process to compress the material one or more times using cold rolls and to compress the material one or more times using hot rolls. In the context of the present invention, hot means temperatures in the range from 100 to 250° C., preferably from 120 to 200° C.

The present invention also provides modified foams obtainable by the inventive process, these also being termed inventive foams below.

Inventive modified foams have a density in the range from 5 to 1,000 kg/m³, preferably from 6 to 500 kg/m³, and particularly preferably in the range from 7 to 300 kg/m³. The density of the inventive foam is affected on the one hand via the degree of coating with compound (b) and, if appropriate, with catalyst (c), and, if appropriate, with at least one additive (d), and on the other hand via the degree of compaction of the starting material. Density and rigidity or flexibility can be adjusted as desired via suitable selection of the degree of coating and of compaction.

Inventive modified foams preferably comprise an amount in the range from 0.1 to 80% by weight, preferably from 2 to 60% by weight, particularly preferably from 5 to 50% by weight, based on the weight of the corresponding unmodified foam (a), of solid derived from (b).

Inventive modified foams or foams produced by the inventive process feature properties which are in total advantageous and which eliminate the disadvantages described above, such as short service time, damage to delicate surfaces, and unsightly appearance. They exhibit improved cleaning performance or cleaning action, good resistance to hydrolysis, improved resistance to acids, and good sound absorption and—for example if they are used to produce cleaning materials—are particularly durable. They last for long periods without soiling. In the event that inventive foams become soiled, they can readily undergo non-destructive cleaning. Another feature of inventively modified foams or of inventive modified foams is high resistance to oxidants, in particular to gaseous oxidants, such as ozone and oxygen. Inventive modified foams are moreover highly flexible and can easily be converted mechanically to desired shapes. Furthermore, inventive modified foams have an attractive cloth-like feel and are particularly non-aggressive when cleaning delicate surfaces.

Inventive modified foams are moreover suitable for applications in the cosmetics sector, for example as towels or pads for make-up removal, or for hygiene products.

Inventively modified foams are particularly advantageous in any of the applications where flexibility of the material is required.

The present invention also provides the use of inventive modified open-cell foams or of inventively modified open-cell foams for production of cleaning materials, such as wipers, brushes, wiper cloths, wiper mops, cleaning cloths, cleaning granules, or oil-absorbent materials, for example for manual or machine cleaning, cleaning materials in the form of filamental materials, if appropriate in a composite with filaments or wires composed of other materials, e.g. polyamide or metal, these being suitable core materials for the cleaning of, for example, eyelets, of drawing dies, of screw threads, or of spindels,
filters, such as air filters, pond filters, aquarium filters, water filters, or else as a matrix for ceramic filters,
humidifers, water distributors,
   packaging elements, in particular for impact- or water-sensitive products, vibration-damping elements, sound-deadening elements, buildings-insulation materials, in particular roof-insulation materials and wall-insulation materials.

The present invention also provides a process for production of cleaning materials, using inventive modified foams or using inventively modified foams. The present invention also provides a process for production of filters, using inventive modified foams, or using inventively modified foams. The present invention also provides a process for production of humidifiers, using inventive modified foams, or using inventively modified foams. The present invention also provides a process for production of cosmetics items, using inventive modified foams, or using inventively modified foams. The present invention also provides a process for production of water distributors, using inventive modified foams, or using inventively modified foams. The present invention also provides a process for production of packaging elements, using inventive modified foams, or using inventively modified foams. The present invention also provides a process for production of sound-deadening elements, using inventive modified foams, or using inventively modified foams. The present invention also provides a process for production of buildings-insulation materials, using inventive modified foams, or using inventively modified foams.

If the intention is to use inventive modified foams for production of filters, preference is given to sack filters and matrices of ceramic filters. If the intention is to use inventive modified foams for production of automobile parts, ventilation units are particularly preferred.

The present invention also provides cleaning materials, filters, humidifiers, cosmetics items, water distributors, packaging elements, sound-deadening elements, and buildings-insulation materials produced using, or comprising, inventive modified foams or inventively modified foams.

By way of example, inventive modified foams may be connected, for example mechanically, to other materials, for example to poles, bases for, by way of example, brooms and brushes, or to textiles, leather, polymers such as polyurethane, or wood.

Inventive modified foams give good results when printed, for example by the ink-jet process, or using pigmented printing pastes.

Inventive modified foams can, for example, perform well in application to supports, which can be curved or flat, rigid or flexible. Examples of supports are textile supports, paper supports, nets, and also plastic sheets and metal sheets. A particular embodiment that may be mentioned is application to textile supports for manual use. Inventive modified foams perform particularly well in application to textile supports for belt grinding machines, vibratory sanders and/or polishing disks. The invention therefore further provides a process for applying inventive modified foams to supports, which can be curved or flat, rigid or flexible, in particular to textile supports or to paper supports. Examples of methods of inventive application are adhesive-bonding, sewing, or riveting.

The present invention further provides composites, comprising at least one inventive modified foam and at least one support which by way of example can be curved or flat, rigid or flexible, examples being a textile support or paper support. The present invention further provides the use of inventive modified foams, applied to abovementioned supports, in particular to textile supports or to paper supports, as a tool for belt grinding machines and vibratory sanders or for manual use, for example in the form of polishing disks.

The invention is illustrated via examples,

### EXAMPLES

I.1 Production of Unmodified Foam (a)

A spray-dried melamine-formaldehyde precondensate (molar ratio 1:3, molar mass about 500 g/mol) was added, in an open vessel, to an aqueous solution with 3% by weight of formic acid and 1.5% of the sodium salt of a mixture of alkylsulfonates having from 12 to 18 carbon atoms in the alkyl radical (K 30 emulsifier from Bayer AG), the percentages being based on the melamine-formaldehyde precondensate. The concentration of the melamine-formaldehyde precondensate, based on the entire mixture composed of melamine-formaldehyde precondensate and water, was 74% by weight. The resultant mixture was vigorously stirred, and then 20% by weight of n-pentane were added. Stirring was continued (for about 3 min) until a dispersion of homogeneous appearance was produced. This was applied, using a doctor, onto a Teflon-treated glass fabric as substrate material and foamed and cured in a drying cabinet in which the prevailing air temperature was 150° C. The resultant temperature within the foam composition was the boiling point of n-pentane, which was 37.0° C. under these conditions. After from 7 to 8 min, the foam had risen to its maximum height. The foam was then left for a further 10 min at 150° C. in the drying cabinet; it was then heat-conditioned for 30 min at 180° C. This gave unmodified foam (a.1).

The following properties were determined on the unmodified foam (a.1) from Example I.1:
open-cell factor 99.6% to DIN ISO 4590,
compressive strength (40%): 1.3 kPa determined to DIN 53577,
density: 7.6 kg/m³ determined to EN ISO 845,
average pore diameter: 210 μm, determined via evaluation of micrographs of sections,
BET surface area: 6.4 m²/g, determined to DIN 66131,
sound absorption: 93%, determined to DIN 52215,
sound absorption: above 0.9, determined to DIN 52212.

I.2 Production of Inventive Modified Foams

Unmodified foam (a.1) from Example I.1 was cut to give foam blocks of dimensions 9 cm·4 cm·4 cm. The weight of the foam blocks was in the range from 1.00 to 1.33 g. Pieces of unmodified foam with weight as in Table 1 were then brought into contact with an aqueous dispersion comprising 81 g/l of N,N′-dimethyl-4,5-dihydroxyimidazolinone (I b.1) and 18 g/l of MgCl₂.6H₂O,

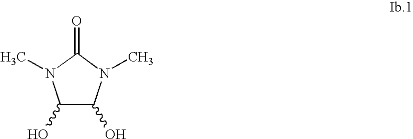

by completely immersing each foam block in the aqueous dispersion and allowing it to stand for 2 minutes with a covering of aqueous dispersion. The foam block was then removed from the relevant aqueous dispersion and excess aqueous dispersion was removed by squeezing, by passing the material through two counterrotating rolls with diameter of 150 mm and separation of 8 mm, rotating at 32 rpm. The liquor absorption thus achieved was 520% by weight.

The material was then dried for a period of 4 hours at 80° C. in a drying cabinet. Heat-setting was then carried out at 150° C. for 10 minutes in the drying cabinet. This gave inventive modified foam F1.1.

II. Production of Other Inventive Modified Foams

II.1 Production of Inventive Modified Foam F1.2

The experiment in I.2 was repeated, but the material was brought into contact with an aqueous dispersion of
120 g/l of (I b.1), and
57.8 g/l of MgCl₂.6H₂O

The foam block was removed 5 seconds after immersion, and squeezed as described above to remove material, the liquor absorption achieved being 540% by weight.

Heat-setting was then carried out with no prior drying for 15 minutes at 150° C. in the drying cabinet.

This gave inventive modified foam F1.2.

II.2 Production of Inventive Modified Foams F2.1

Blocks (dimensions: 9 cm·4 cm·4 cm ) of unmodified foam (a.1) were sprayed with an aqueous dispersion, comprising
112.5 g/l of (I b.2), and
61.4 g/l of MgCl₂.6H₂O.

The materials were allowed to interact for 2 minutes, and then squeezing to remove excess material was carried out as described in 1.2, and heat-setting was carried out for 20 minutes at 140° C. in a drying cabinet. The resultant liquor absorption was 425% by weight.

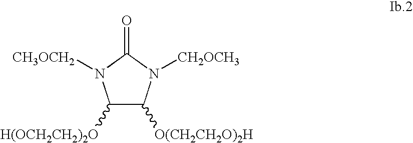

This gave inventive modified foam F2.1.

II.3 Production of Inventive Modified Foams

Pieces of unmodified foam from Example I.1 with weight as in Table 1 were brought into contact with an aqueous dispersion comprising
112.5 g/l of (I b.2), and
61.4 g/l of MgCl₂.6H₂O,
   by completely immersing each foam block in the aqueous dispersion and allowing it to stand for 2 minutes with a covering of aqueous dispersion. The foam block was then removed from the relevant aqueous dispersion and excess aqueous dispersion was removed by squeezing, by passing the material through two counterrotating rolls with diameter of 150 mm and separation of 5 mm, rotating at 32 rpm. The liquor absorption thus achieved was 110% by weight.

The material was then dried for a period of one hour at 80° C. in a drying cabinet. Heat-setting was then carried out at 160° C. for 10 minutes in the drying cabinet. This gave inventive modified foam F2.2.

II.4 Production of Inventive Modified Foam F2.3

Pieces of unmodified foam from Example I.1 with weight as in Table 1 were brought into contact with an aqueous dispersion comprising
60 g/l of (I b.2), and
25 g/l of MgCl₂.6H₂O,
   by completely immersing each foam block in the aqueous dispersion and allowing it to stand for 2 minutes with a covering of aqueous dispersion. The foam blocks were then removed from the relevant aqueous dispersion and excess aqueous dispersion was removed by squeezing, by passing the material through two counterrotating rolls with diameter of 150 mm and separation of 8 mm, rotating at 32 rpm. The liquor absorption thus achieved was 725% by weight.

Heat-setting was then carried out at 150° C. for 10 minutes in the drying cabinet (without prior drying). This gave inventive modified foam F2.3.

II.5 Production of Inventive Modified Foam F2.4

The procedure was as described in Example II.4, but the material was dried for 2 hours at 80° C. in a drying cabinet prior to the setting process and was heat-set for 5 minutes at 180° C. This gave inventive modified foam F2.4.

Liquor absorption was determined prior to drying, the value obtained being 450% by weight.

TABLE 1
Inventive modified foams (data in % by weight, based on weight of
unmodified foam)

| | Inventive | Weight of | | |
|---|---|---|---|---|
| | modified | unmodified foam | Weight of inventive | Δ [% by |
| (b) | foam No. | block [g] | modified foam [g] | weight] |
| (b I.1) | F1.1 | 1.09 | 1.57 | 44 |
| (b I.1) | F1.2 | 1.21 | 2.0 | 65 |
| (b I.2) | F2.1 | 1.13 | 1.67 | 48 |
| (b I.2) | F2.2 | 1.22 | 1.37 | 12 |
| (b I.2) | F2.3 | 1.15 | 1.65 | 43 |
| (b I.2) | F2.4 | 1.11 | 1.41 | 27 |

III. Use of Inventive Modified Foams and of Unmodified Foams as Cleaning Cloths

Inventive modified foams and unmodified foam were in each case used as wipers.

In each case, inventive modified foams and unmodified foam were moistened with water.

The material cleaned in each case manually for a period of 2 minutes using one of the inventive modified foams from I.2 or II. and using unmodified foam of I.1, was about 1 m² of a painted plasterboard wall (rough) which had been soiled with streaks of abraded rubber, shoe cream, and used oil. This gave cleaned walls as in Table 2, and the cleaning quality of these was assessed visually. The dimensional stability of the wipers was also assessed visually.

TABLE 2
Unmodified foam (a.1) from I.1, inventive modified foams, and their
use as wipers

| Foam | Cleaning quality | Dimensional stability of foam |
|---|---|---|
| (a.1) | satisfactory | marked loss of shape after 2 minutes |
| (b I.1) | very good | no loss of shape |
| (b I.1) | very good | no loss of shape |
| (b I.2) | very good | no loss of shape |
| (b I.2) | good | slight loss of shape |
| (b I.2) | very good | no loss of shape |
| (b I.2) | very good | slight loss of shape |

IV. Production of Other Inventive Modified Foams

Unmodified foam (a.1) from Example I.1 was cut to give foam blocks with dimensions 10 cm·10 cm·0.5 cm. The weight of the foam blocks was in the range from 0.35 to 0.48 g.

IV.1 Production of Inventive Modified Foam F1.3

A foam block from IV. with weight 0.44 g was brought into contact with an aqueous dispersion of
81 g/l of (b I.1), and
18 g/l of MgCl₂.6H₂O,
   by completely immersing each foam block in the aqueous dispersion and allowing it to stand for 2 minutes with a covering of aqueous dispersion. The foam blocks were then removed from the relevant aqueous dispersion and excess aqueous dispersion was removed by squeezing, by passing the material through two counterrotating rolls with diameter of 150 mm and separation of 2 mm, rotating at 32 rpm. The liquor absorption thus achieved was 420% by weight.

The material was then dried for a period of 4 hours at 80° C. in a drying cabinet. Heat-setting was then carried out at 150° C. for 10 minutes in the drying cabinet. Based on unmodified foam, the resultant amount of coating was 34% by weight.

The treated foam was then moistened with water and calendered by passing it ten times through two counterrotating rolls subjected to a pressure of 3.5-4 bar, whereupon the treated foam was compressed (mechanically) to about one third of its initial thickness. This gave inventive modified foam F1.3, which had an attractive soft cloth-like feel and was flexible.

IV.2 Production of Inventive Modified Foam F1.4

A foam block from IV. with weight 0.44 g was brought into contact with an aqueous dispersion of
81 g/l of (b I.1), and
18 g/l of MgCl₂.6H₂O,
   by immersing it completely in the aqueous dispersion. After 5 seconds, the foam blocks were then removed from the relevant aqueous dispersion and excess aqueous dispersion was removed by squeezing, by passing the material through two counterrotating rolls with diameter of 150 mm and separation of 2 mm, rotating at 32 rpm. The liquor absorption thus achieved was 420% by weight.

Calendering was then carried out by passing the treated foam twelve times through two counterrotating rolls subjected to a pressure of 3.5-4 bar, whereupon the treated foam was compressed (mechanically) to about one third of its initial thickness. Heat-setting was then carried out for 15 minutes at 150° C. in a drying cabinet. Based on unmodified foam, the resultant amount of coating was 34% by weight. This gave inventive modified foam F1.4, which had an attractive soft cloth-like feel and was flexible.

IV.3 Production of Inventive Modified Foam F2.5

A foam block from IV. with weight 0.48 g was sprayed with 3.9 times its weight of an aqueous dispersion comprising
112.5 g/l of (I b.2), and
61.4 g/l of MgCl₂.6H₂O.

The materials were allowed to interact for 2 minutes, and then excess aqueous dispersion was removed by squeezing, by passing the material through two counterrotating rolls with diameter 150 mm and separation 2 mm, rotating at 32 rpm.

The resultant liquor absorption was 325% by weight and the resultant amount of coating was 37% by weight.

The material was then heat-set in a drying cabinet for 15 minutes at 150° C. (without prior drying).

The treated foam was then moistened with water and calendered, by passing it fifteen times through two counterrotating rolls subjected to a pressure of 3.5-4 bar, whereupon the treated foam was compressed (mechanically) to about 40% of its initial thickness. This gave inventive modified foam F2.5, which had an attractive soft cloth-like feel and was flexible.

IV.4 Production of inventive modified foam F2.6

A foam block from IV. with weight 0.42 g was brought into full contact with an aqueous dispersion comprising
112.5 g/l of (I b.2), and
61.4 g/l of MgCl₂.6H₂O,
   by immersing it completely in the aqueous dispersion. The materials were allowed to interact for 2 minutes, and then excess aqueous dispersion was removed by squeezing, by passing the material through two counterrotating rolls with diameter 150 mm and separation 2 mm, rotating at 32 rpm. The resultant liquor absorption was 360% by weight and the resultant amount of coating was 41% by weight.

The material was then dried for one hour at 80° C. in a drying cabinet and was heat-set in the drying cabinet for 7.5 minutes at 160° C.

The treated foam was then moistened with water and mechanically compressed by compressing it ten times, using a plate press, to about one third of its initial thickness. This gave inventive modified foam F2.6, which felt like soft leather and was highly flexible.

IV.5 Production of Inventive Modified Foam F2.7

A foam block from IV. with weight 0.46 g was brought into contact with an aqueous dispersion comprising
60 g/l of (I b.2), and
25 g/l of MgCl₂.6H₂O,
   by immersing it completely in the aqueous dispersion and allowing it to stand for 2 minutes with a covering of aqueous dispersion. The foam blocks were then removed from the relevant aqueous dispersion and excess aqueous dispersion was removed by squeezing, by passing the material through two counterrotating rolls with diameter of 150 mm and separation of 2 mm, rotating at 32 rpm. The liquor absorption thus achieved was 725% by weight.

The material was then heat-set in a drying cabinet for 10 minutes at 150° C. (without prior drying).

The treated foam was then moistened with water and calendered, by passing it ten times through two counterrotating rolls subjected to a pressure of 3.5-4 bar, whereupon the treated foam was compressed (mechanically) to about one third of its initial thickness. This gave inventive modified foam F2.7, which had an attractive soft cloth-like feel and was flexible.

IV.6 Production of Inventive Modified Foam F2.8

A foam block from IV. with weight 0.35 g was brought into contact with an aqueous dispersion comprising
60 g/l of (I b.2), and
25 g/l of MgCl₂.6H₂O,
   by immersing it completely in the aqueous dispersion and allowing it to stand for 2 minutes with a covering of aqueous dispersion. The foam blocks were then removed from the relevant aqueous dispersion and excess aqueous dispersion was removed by squeezing, by passing the material through two counterrotating rolls with diameter of 150 mm and separation of 2 mm, rotating at 32 rpm. The liquor absorption thus achieved was 710% by weight.

The material was then first dried for 2 hours at 80° C. in a drying cabinet. It was then heat-set in the drying cabinet for 5 minutes at 180° C.

The treated foam was then moistened with water and mechanically compressed by compressing it ten times, using a plate press, to about one third of its initial thickness. This gave inventive modified foam F2.8, which felt like soft chamois leather and was highly flexible.

V. Use of Inventive Modified Foam from IV. and of Unmodified Foams as Cleaning Cloths

Inventive modified foams and unmodified foam were in each case used as cleaning cloths for cleaning of a delicate surface composed of Plexiglas.

Circular disks (diameter: 4.5 cm), each of thickness of about 0.5 cm, were cut from unmodified foam (a.1) and from inventively modified foam from IV., and adhesive-bonded to a weight (about 1600 g). This gave test specimens. The test specimens were slightly moistened with water and rubbed about 2000 times across Plexiglas, with the aid of a “Prüfbau-Quant-Scheuerprüfer”. In order to assess whether the inventive foams caused less detrimental scratching than the untreated foam on the surface of the Plexiglas, the scratches on the rubbing area were counted under magnification (1:75) provided by a microscope.

The average number of scratches caused by the untreated foam (a.1) was 31;
using F1.4 9 scratches,
using F2.5 2 scratches,
using F2.7 7 scratches.

VI. Use of Inventive Modified Foams as Belt

Inventive modified foam I.2 was adhesive-bonded to a piece of linen textile with dimensions 5 cm·5 cm with the aid of an adhesive based on silicone rubber. This gave an inventive composite. The inventive composite was stored for 24 hours, moistened with water, and used for 10 seconds of manual cleaning of a coin (1 Euro cent piece). The coin was then cleaned, and the strike had suffered no scratch damage through the cleaning process.

The inventive composite withstood repeated manual buckling and crumpling without damage.

1. A process for production of modified open-cell aminoplastic foams) which comprises bringing the following materials into contact
(a) open-cell aminoplastic foams with density in the range from 5 to 500 kg/m³ and average pore diameter in the range from 1 μm to 1 mm, and
(b) aqueous formulation of at least one compound having at least one hemiaminal or aminal group per molecule, or at least one copolymer comprising at least one (co)monomer of the formula II 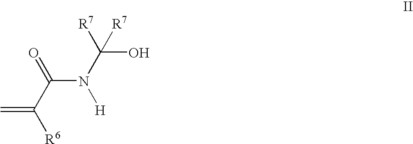
the selected variables being as follows:
R⁶ selected from hydrogen and C₁-C₁₂-alkyl
R⁷ different or identical and selected from C₁-C₁₂-alkyl and hydrogen.
or β-dicarbonyl-containing or epoxy-containing comonomer,

2. The process according to claim 1, wherein at least one compound from step (b) has not been used during production of open-cell aminoplastic foam (a).

3. The process according to claim 1, wherein compound (b) is obtained via condensation of at least one nitrogen-containing compound (B 1) and of at least one carbonyl compound (B2), and, if appropriate, of other compounds (B3), and, if appropriate, further reactions after the condensation process.

4. The process according to claim 1, wherein the materials are also brought into contact with
(c) at least one catalyst.

5. The process according claim 1, wherein the materials are also brought into contact with at least one additive (d), these additives (d) being selected from biocides, surfactants, activated charcoal, colorants, fragrances, odor scavengers, and microcapsules, charged with at least one active ingredient.

6. The process according claim 1, wherein, after open-cell aminoplastic foam (a) has been brought into contact with aqueous formulation of compound (b), and, if appropriate, with catalyst (c), and, if appropriate, with at least one additive (d) the materials are allowed to interact and mechanical compression is then carried out.

7. The process according to claim 1, wherein, after open-cell aminoplastic foam (a) has been brought into contact with aqueous formulation of compound (b), and, if appropriate, with catalyst (c), and, if appropriate, with at least one additive (d) the materials are allowed to interact and heat-setting is then carried out.

8. The process according to claim 1, wherein, after open-cell aminoplastic foam (a) has been brought into contact with aqueous formulation of compound (b), and, if appropriate, with catalyst (c), and, if appropriate, with at least one additive (d) the materials are allowed to interact, and mechanical compression and heat-setting are then carried out.

9. The process according to claim 1, wherein the open-cell aminoplastic foams (a) are melamine foams.

10. The process according to claim 1, wherein, in step (b), the materials are brought into contact with at least one compound of the general formula I a to I b 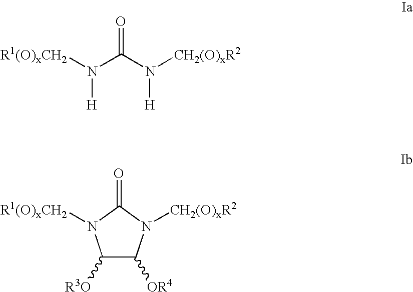
the variables being defined as follows:
R¹ and R² are identical or different and are selected from hydrogen, C₁-C₁₂-alkyl, branched or unbranched, (—CH₂—CH₂—O)ₘ—R⁵, (—CHCH₃—CH₂—O)ₘ—R⁵, (—CH₂—CHCH₃—O)ₘ—R⁵, (—CH₂—CH₂—CH₂—O)ₘ—R⁵, (—CH₂—CH₂—CH₂—CH₂—O)ₘ—R⁵,
x are identical or different and are a whole number selected from zero and one, at least one x in formula I a being selected to be equal to 1,
m is a whole number in the range from 1 to 20,
R³ and R⁴ are identical or different and are selected from hydrogen, C₁-C₁₂-alkyl, branched or unbranched, or together are C₂-C₄-alkylene,
R⁵ are identical or different and are selected from C₁-C₄-alkyl and hydrogen.

11. A modified open-cell aminoplastic foam, obtainable by a process according to claim 1.

12. (canceled)

13. A process for production of cleaning materials, of filters, of humidifiers, of cosmetics items, of water distributors, of packaging elements, of sound-deadening elements, or of buildings-insulation materials, wetting the modified open-cell aminoplastic foams, produced by a process according to claim 1 and mechanically compressing the foam.

14. A cleaning material, a filter, a humidifier, a cosmetics item, a water distributor, a packaging element, a sound-deadening element, or a buildings-insulation material, produced using modified open-cell aminoplastic foams, produced by a process according to claim 1.

15. A cleaning material, a filter, a humidifier, a cosmetics item, a water distributor, a packaging element, a sound-deadening element, or a buildings-insulation material, comprising modified open-cell aminoplastic foams, produced by a process according to claim 1.

16. A process for applying modified open-cell aminoplastic foams according to claim 11 to a curved or flat, rigid or flexible support.

17. A composition, comprising at least one modified open-cell aminoplastic foam according to claim 12 to a curved or flat, rigid or flexible support.

18. A process of treating a sample comprising wetting the foam of claim 11, and rubbing the sample.
